(19) United States
(12) Patent Application Publication
(10) Pub. No.: US 2006/0014161 A1
(43) Pub. Date: Jan. 19, 2006

(54) Combination comprising biochip and optical detection device

(76) Inventors: Olfert Landt, Berlin (DE); Volker Heiser, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/991,078

(22) Filed: Nov. 18, 2004

(30) Foreign Application Priority Data
Nov. 18, 2003 (DE) 203 17 944.7

(51) Int. Cl. C12Q 1/68; G01N 33/53; C12M 1/34

(52) U.S. Cl. 435/6; 435/7.92; 435/287.2

(57) ABSTRACT

The invention relates to a method and apparatus for the routine analysis of samples that contain biomolecules. The invention uses biochips or arrays in the format of conventional 35 mm film slides. These film slide arrays allow employing commercially available detection, storage and processing equipment. The use of array technology is thus greatly facilitated for applications where cost of equipment and ease of handling are key factors.

### FIELD OF THE INVENTION

The present invention relates to the field of medical and biological diagnosis. In particular, it concerns the field of biochips for molecular diagnostics.

### BACKGROUND OF THE INVENTION

Biochip technology has become an important field of technological development. Within the context of this disclosure, biochip technology is meant to comprise the plurality of methods based on attaching biological molecules to a surface and analyzing the interaction of these attached molecules with a sample. Biological molecules in this context are nucleic acids, peptides, proteins, small molecules from the realm of organic chemistry, and related structures.

Such biochips facilitate the parallel analysis of a sample with different reactants or biological molecules.

Reviews of the current state of the art in the field of biochips are those of Ng and IIag (Biotechnol Annu Rev. 2003; 9:1-149) or Jain (Curr Opin Drug Discov Devel. 2004, 7(3):285-289) and the citations contained therein, which are incorporated by reference.

The best-known biochip technology is the attachment of nucleic acids to surfaces. Such attachment results in “DNA chips”. Two-dimensional arrangements (arrays) of nucleic acids can be used to analyze a great number or even all of the genes (genome) or transcripts (transcriptome) contained in a cell. Since scientific interest has focused more and more on problems where the parallel analysis of ever more parameters is important, array technology has concentrated on the development of ever more complex chips with more different attached biomolecules. This is also true for the trend in peptide and protein chips.

The development of arrays with higher density and complexity has immensely increased their capacity; the costs for production and processing of these chips, especially for the complex optical or electronic equipment used for reading them, have increased as well.

While research labs continue to demand chips of higher density, more and more applications of chip technology for routine analysis in clinical microbiology, food safety and forensics are developed. Examples are typing of microbial agents and resistance in bacteria, the absence or presence of certain meat ingredients or the presence of genetically modified organisms or genetic fingerprinting.

In many cases, these applications do not need high-density arrays comprising thousands of immobilized ligands, but typically below one hundred different attached ligands for sample ingredients. Rather than high density, routine applications call for reproducibility, simplicity of procedure and, most importantly, the cost of the analysis are of importance in routine applications. With regard to the cost aspect, not only the production cost for the chip and the reagents necessary for processing are important here, but also the equipment cost. Many chip readers are very complex and expensive investment items. Such equipment will be affordable for routine analysis only in exceptional cases.

The technology for the production and processing of biochips is known in the art:

Different carrier materials for the biomolecules are known, for example glass, thermoplastic polymers, crystalline or amorphous silicon, gold-plated glass or aluminum.

For attachment of the biomolecules, different methods are used. The binding to the surface of the biochip can be attained by adsorption, covalent coupling to activated surfaces or binding of accordingly activated molecules to native surfaces.

The detection of a sample molecule-binding event can be effected by a number of ways. The binding of a nucleic acid usually is caused by a sequence-specific interaction of the nucleic acid molecule to another nucleic acid molecule or an analogue attached to the surface. Such binding usually is detected most commonly by fluorescence detection of bound sample molecules that had been marked with fluorescent dyes prior to binding, or which are detected after binding to the array by a third reagent (see the citation of Jain, ibid.). The detection can also be effected enzymatically or by means of radioactive marker detection.

The detection of changes in the fluorescence activity of the chip or sample is relatively difficult and equipment-intensive. Taton et al. (Science 289, 1157-1161 (2000); U.S. Pat. No. 6,361,944) describe the detection of nucleic acids linked to gold nanoparticles. The presence of bound sample molecules is detected by reduction of silver (I) ions to metallic silver in visible light. Conventional document scanners can thus be employed to achieve a densitometric analysis. The authors of said publication assert to have achieved an increase in assay sensitivity of at least two orders of magnitude in comparison to similar fluorescence detection systems.

In this and most other applications of array technology, the format of the array is the conventional sample support known from microscopy. The size of such sample support or microscopy slide is 1×3 inch (7.5×2.5 cm), with a thickness of 0.5 to 1.5 mm. Glass has historically been the point of departure for the chemistry to attach biomolecules on the array. With increasing complexity of chips, the single points representing one attached biomolecule population became ever smaller. As a consequence, microscopes had to be employed as the detection device. This has further accelerated the trend towards highly complex, expensive detection equipment. High-density chips are commonly read by confocal microscopes. The cost of such equipment is between 30,000 and more than 200,000 US$ (as of 2003).

The only exception known to us is the reading of conventional microscopy slides by flatbed optical scanners as commercialized by Nanosphere Inc. or ArrayIt Inc. The handling of microscopy slides entails disadvantages relating to the automatisation of the production and the processing of slides, which makes an improvement of that technology desirable. Particularly, the resolution of commercial flatbed scanners is too low at present to allow quantitative measurements of optical density data on biomolecule arrays. Furthermore, an array arranged on a conventional microscopy slide must be positioned and precisely fixed in the correct orientation, by hand, to make the resulting image amenable to computerized analysis; this criterion is added regardless of the insufficient resolution of the flatbed scanner.

Materials, coupling methods for biomolecules to surfaces and applications thereof are well known in the art; one example is the German patent DE 1032104 and its international family members, incorporated herein by reference.

Departing from this state of the art, it is the objective of the present invention to provide a combination of a biochip and a detection device. Said combination allows an economically viable production and processing of biomolecule arrays for their use in routine analysis.

### GENERAL DESCRIPTION OF THE INVENTION

According to the present invention, said objective is attained by choosing a format for the biochip that corresponds to the frame size of conventional 35 mm diapositive slide frames of 50 mm by 50 mm. Furthermore, an important element of the present invention is the use of diapositive slide scanners or—readers that digitize the optical information contained on the array in film slide format, making it amenable to computerized analysis.

An important advantage of the invention is the aspect that the array in film slide format (50 mm×50 mm) can be handled and read in automated form by stacks or magazines. Furthermore, a wide range of accessories for archiving, transporting and cleaning of the arrays can be purchased and used with ease. Another advantageous aspect is the fact that the arrays can be visualized by commercially available slide projectors.

Another advantage of the invention is that commercially available detection devices for the inventive arrays, that is slide scanners and electronic cameras with mounting devices for 35 mm film diapositives, show a much better resolution and better uniformity of illumination when compared to flatbed scanners, which allows the quantitative analysis of results obtained with silver reduction or other array staining methods.

Another advantage of the invention is that the reproducibility of analyses of the inventive array format is much greater when handled in automated fashion, in comparison to microscopy slide arrays handled on flatbed scanners manually or with the aid of masks.

Furthermore, the inventive array can be made or processed in machines and devices used for photograph processing and development, which makes additional development of apparatus unnecessary, and hence makes the overall use of the inventive arrays more attractive commercially.

Furthermore the invention facilitates the handling of conventional microscopy slides by providing an adapter having the 35 mm diapositive film slide format of 50×50 mm in combination with the corresponding reading devices.

### DETAILED DESCRIPTION OF THE INVENTION

The inventive array or biochip has an essentially square format of 50 mm by 50 mm (+/−2 mm) and a thickness of maximally 2 mm (+0,5 mm/−1 mm). The surface of the inventive array or biochip can be uniformly coated or divided into a central functional surface with rectangular dimensions of maximally 36 mm by 24 mm, and an outer frame surface. According to one preferred embodiment, the functional surface comprises several functional zones. In another preferred embodiment, one functional zone covers the entire functional surface.

According to one preferred embodiment, the entire surface of the array or biochip is transparent for all wavelengths of visible light in order to allow for optimal illumination of the array. In another preferred embodiment, the surface of the array is transparent but colored in order to achieve a filtering effect. In yet another preferred embodiment the surface is semi-transparent or opaque, in order to allow for a uniform illumination when illuminating the array from a non-uniform light source such as a lightbulb. In yet another preferred embodiment the surface is non-transparent. This enables the reading of fluorescence or reflection, chemo- and bioluminescence.

The functional surface can be partitioned by rounded or square elevated or indented elements into different functional zones. Said elevated or indented elements facilitate the handling of liquids on the array or biochip.

The surface of the functional surface or the entire surface of the chip is chemically modified in the most preferred embodiment. The chemical modification consists of free amino, aldehyde, carboxy, epoxy, hydroxy or sulfhydryl groups or combinations of some or all of the above. Such modification allows for the attachment or immobilization of molecules in form of surface-covering or array-forming functional zones. In one preferred embodiment, the surfaces are covered with a gel, resulting in three-dimensional arrangements in microscopical dimensions.

In one preferred embodiment, synthetic DNA oligonucleotides or products of the Polymerase Chain Reaction (PCR) or Ligase Chain Reaction (LCR) are attached to the surface of the biochip covalently or non-covalently for the purpose of analysis of nucleic acids. For this purpose, the surface of the chip is activated by a suitable method in a first step. Then, oligonucleotides or PCR products are placed onto the chip in a second step. The oligonucleotides or PCR products can be provided as a solution or suspension and be dispensed by means of a stamp, a pipette or by a spraying method. Alternatively, the molecules are attached by irradiation or raising the temperature. Another means of attachment is adhesion through electrostatic interactions. Oligonucleotides or PCR products usually correspond in sequence to nucleic acids that are to be captured in the following step.

The nucleic acids that are to be captured and analyzed in the following step usually are double-stranded sequences that are a product of PCR, but also m-RNA or isolated DNA. These nucleic acids are marked or tagged in a first reaction with a fluorescent marker, biotin, particles or a hapten molecule. The tagging or marking can be achieved covalently or by binding of another correspondingly modified oligonucleotide. A preferred tagging of PCR products is achieved by using tagged primers. The nucleic acid that is to be analyzed is denatured by heating and dispensed onto the surface in a suitable buffer solution, in order to allow complementary sequences to hybridize. In a subsequent washing step, non-bound sequences are removed. The binding of a nucleic acid molecule is analyzed by detection of a fluorescent tag, detection of attached particles or a detection of an attached hapten. The detection can be achieved with or without subsequent amplification. Preferred detection methods are the binding of enzyme-conjugated antibodies that bind to hapten, conjugated streptavidin that binds to biotin and subsequent enzymatic reaction that liberates a dye, or the binding of nanoparticles, preferably particles of gold or silver. These metals can be used as crystallization seeds in a reduction reaction of silver and gold salts that results in an elementary metal precipitate. These reactions, along with enzymatic reactions that result in the precipitation of a dark dye, are preferred for optical detection systems.

### EXAMPLE AND PREFERRED EMBODIMENT

### Streptavidin Coating of a DNA Array

A biochip made of a thermoplastic material and of the format 50×50 mm as provided by the present invention is coated with streptavidin. The chip material is covered for 2 h with a solution of 10 μg/ml streptavidin in PBS buffer (10 mM phosphate, 3 mM potassium chloride, 137 mM sodium chloride).

The chip is washed twice for 1 min in PBS and once in water, and then dried. Further attachments sites are blocked by incubation for one hour with blocking reagent (Roche Diagnostics, used according to the manufacturer's instructions) or milk powder in water (3%). The chip is washed thrice in distilled water and then dried.

Attachment of Specific Probes by Biotin-Streptavidin Linkage

5′-terminally biotinylated DNA oligonucleotide probes are cast in point shape onto the chip surface in a 20 μM solution by means of a stamp or plunger. The plunger has a preferred diameter of 0.05 mm to 0.3 mm. After drying of the oligo probes, the chips are washed twice for one minute in PBS with 0.05% Tween 20, twice in water, and dried. Free streptavidin is saturated with a solution of 0.2 mM solution of biotin in PBS and subsequent washing.

For a DNA array for the identification of meat of different species in food, the following sequences
5′-biotin-TAACAACAATCTCCATgAgTTggT from the 16S RNA region of cattle,
5′-biotin-gATAAAACATAACTTAACATggAC from the 16S RNA region of pig and
5′-biotin-gACCACCTTACAACCTTACACAgC from the 16S RNA region of chicken were spotted to three different points on the biochip and treated as explained above.
   Hybridisation of PCR Products

Homogenized samples of different meat products were agitated for 10 min with a 0.8% solution of NaCl and centrifuged. 2 μl each of the supernatant were given to 10 pmol of the PCR primers 5′-Fluoresceine-TgATCCAACATCgAggTCgTAAACC and 5′-AAgACgAgAAgACCCTRTggARCT, Nucleotides, buffer and Taq polymerase (from the Roche FastStart reagent kit) to a total volume of 20 μl. 45 thermocycles of 5 s at 95° C., 10 s at 60° C. and 5 s at 72° C. are performed. The PCR product is mixed with 10 μl 20× SSC (3 M NaCl, 0.3 M sodium citrate, pH 7.0) buffer and 3 μl 5% SDS, vortexed for 10 sec and incubated on the dry DNA array for 30 min at 50° C. The chip is washed twice for 1 min with 0.2× SSC buffer and dried.

Immunological Detection of Attachment

For detection, 20 μl of a solution of 1 μg/L anti-fluoresceine horseradish peroxidase conjugate (Seramun) is incubated on the chip for 10 min.

The chip is washed 3 times for 1 min in 0.2× SSC buffer and dried. To detect the binding, 20 μl of a solution of TMB (3,3′,5,5′-Tetramethylbenzidine) (Seramun) is brought onto the chip and incubated for 5 to 10 min. The reaction is stopped by washing in water.

Reading of the Analysis Results

The Oxidation of TMB results in a local precipitation of a blue dye at the positions of peroxidase activity. Commercially available dia scanners such as Minolta Dimage Scan 5400, Nikon Supercoolscan 8000 LD etc. can transfer the signals of the inventive biochip or array into image files of different formats, preferably into TIF files in 16 bit grey scale mode. The form (50×50 mm) and the relative positions of reaction zones on the biochip (centrally at 36×24 mm) are of importance here. Conventional software programs for the analysis of biological arrays are, for example, GenePix Pro, Axon Instruments, USA and SlideReader, Chipron GmbH, Germany. These and similar programs can read out image data files and provide an automated analysis with the help of data associating the relative position of probe molecules to the image data.

A qualitative (yes-no) interpretation can thus be achieved (a blue precipitate in the corresponding probe position means that the sausage contained cattle meat). Alternatively, a quantitative analysis can be achieved, since the signal intensity of the image data is correlated to the amount of captured sample molecules.

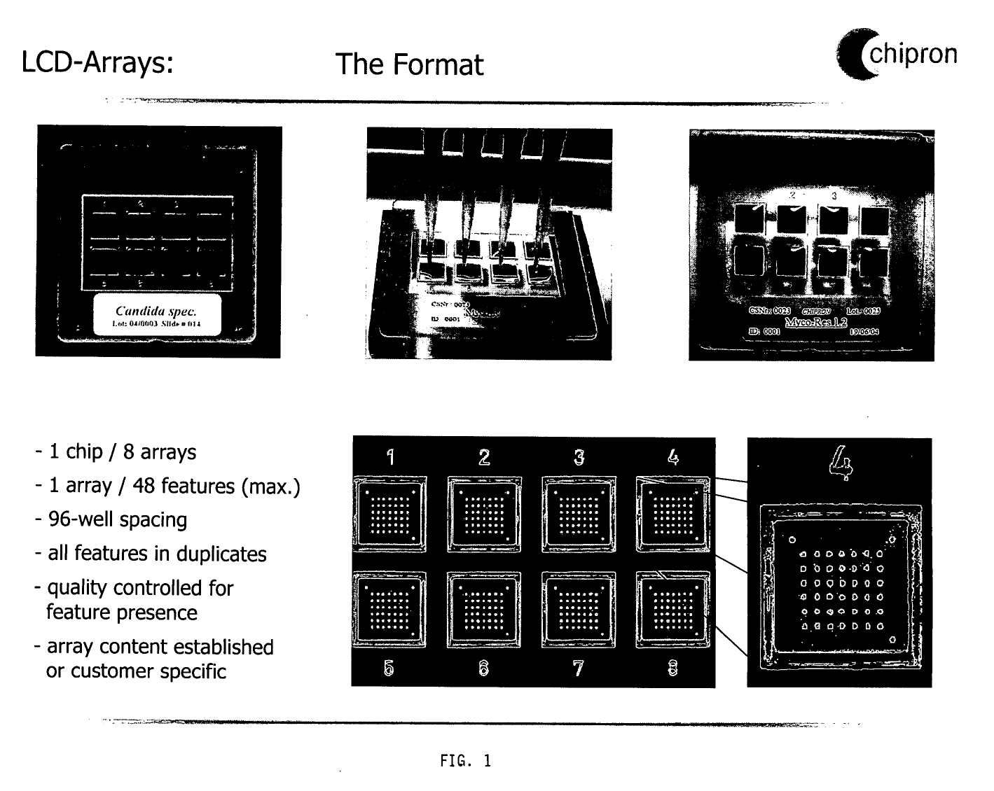

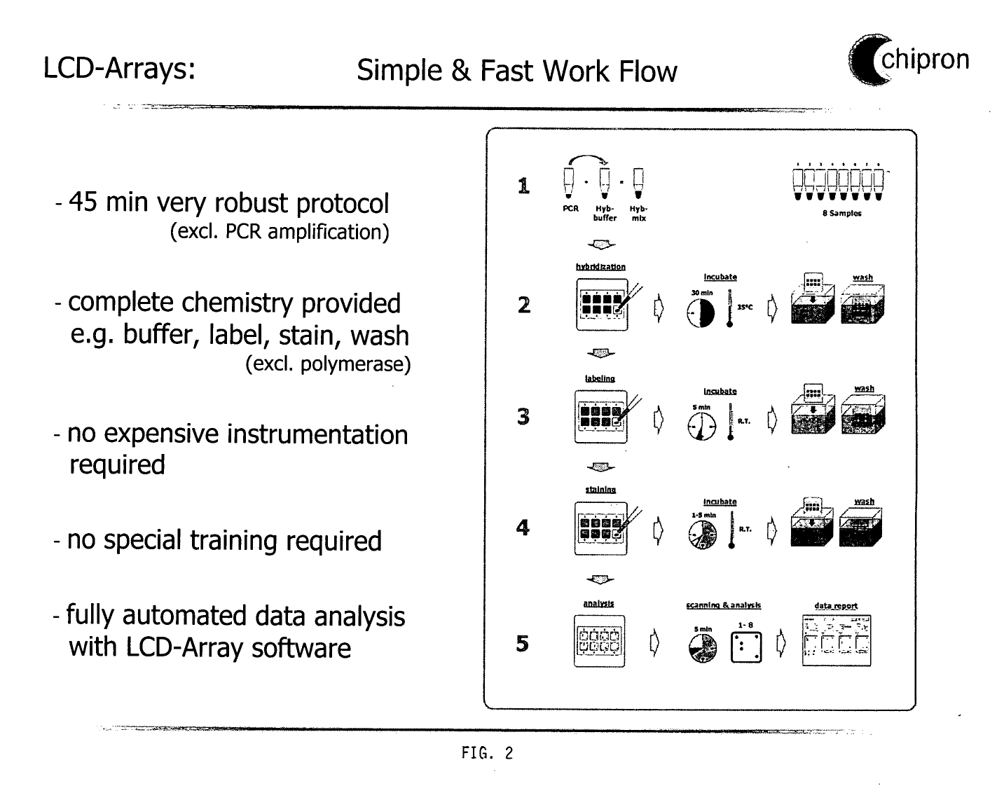

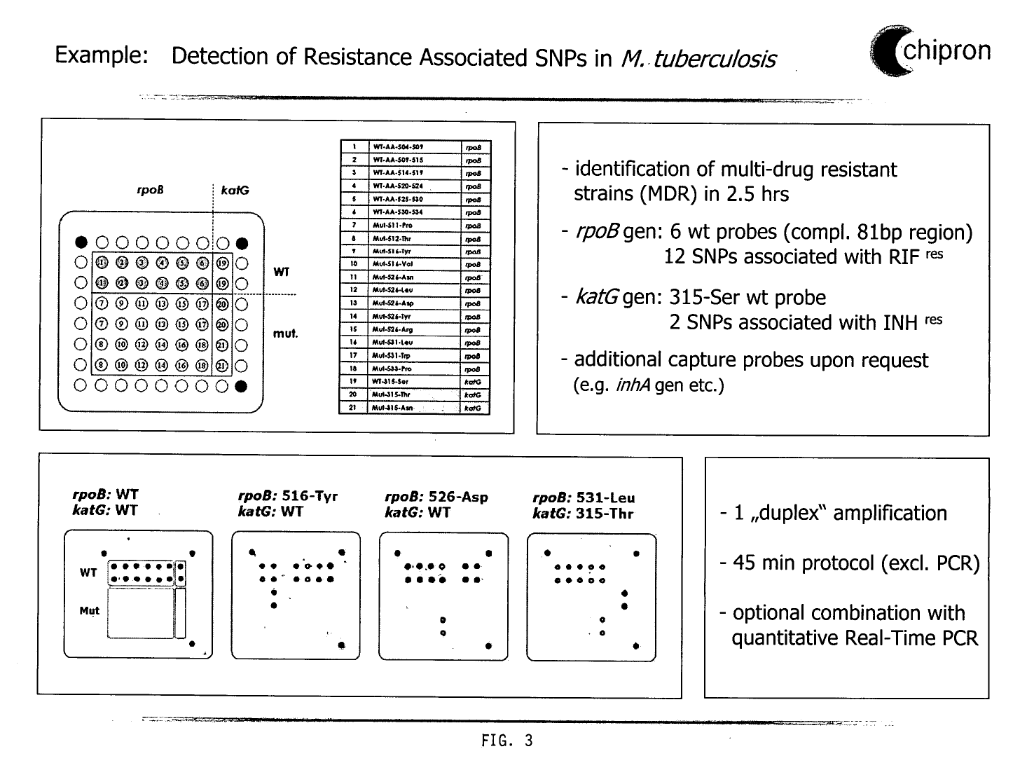

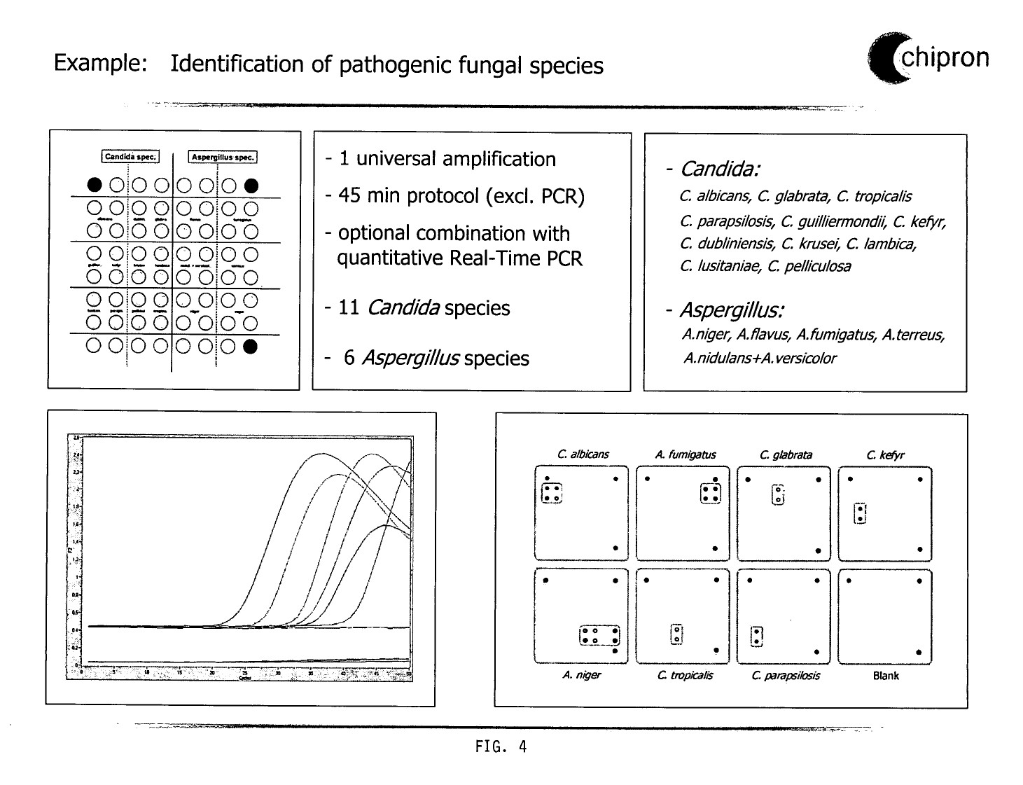

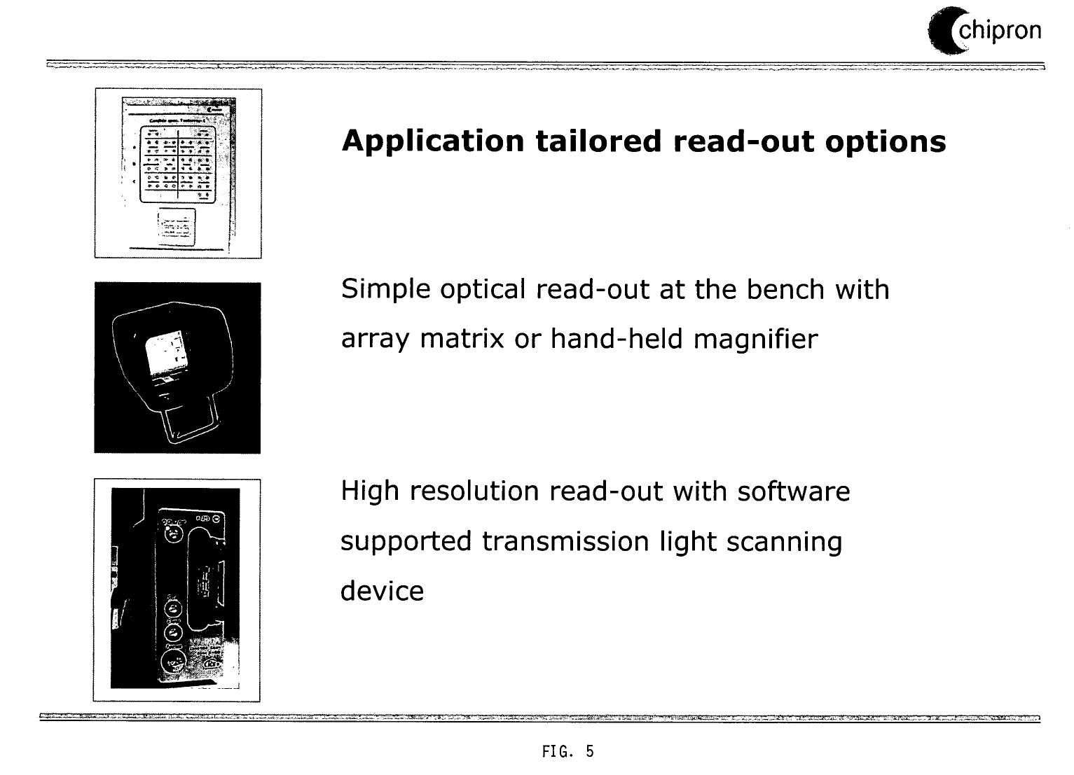

FIGS. 1-5 show an example of uses of the invention.

### SUMMARY

The invention relates to a method and apparatus for the routine analysis of samples that contain biomolecules. The invention uses biochips or arrays in the format of conventional 35 mm film slides. These film slide arrays allow employing commercially available detection, storage and processing equipment. The use of array technology is thus greatly facilitated for applications where cost of equipment and ease of handling are key factors.

What is claimed is:

1. A method for the detection of biomolecules, comprising
a) a first binding step where one or several pluralities of ligand molecules for biomolecules are attached to an essentially flat surface by means of covalent, quasicovalent, ionic, adhesive or adsorptive interaction, where non-attached ligand molecules are then removed by washing and where the thus treated surface is subsequently exposed to a sample containing biomolecules, under conditions that are suitable for the attachment of biomolecules to the ligands attached to said surface, and where non-attached biomolecules are finally removed after exposure to the sample by washing;
b) a second development step where biomolecules attached in the binding step are made discernable by means of a reaction that can be detected in light of the visible wavelengths, and
c) a third detection step where the presence of biomolecules attached in the first binding step detected in an optical image capture system,
characterized by said essentially flat surface having the format of a 35 mm diapositive slide frame of 50 mm by 50 mm and said image capture system being a diapositive film slide scanner with electronic data capture.

2. Method according to claim 1, where the second development step comprises
a) a first enzyme binding step where a suitable enzyme or enzyme conjugate, said enzyme or enzyme conjugate having a binding property specific for attached biomolecules, is brought into contact with said surface, and a second reaction step where a solution of a dye that can be precipitated by said enzyme or enzyme conjugate is brought into contact with said surface, so that a dye precipitate forms at the sites where biomolecules are attached to said surface, or
b) the detection of fluorescence or luminescence by a fluorescently or luminescent active dye attached to said biomolecule by suitable optical or chemical activation, or
c) a metal particle, preferably a gold or silver nanoparticle, being attached to said biomolecule and using said metal particle as a crystallization seed in a subsequent reaction with a gold or silver salt in a reduction reaction, in which a metal precipitate results that is visible in light.

3. Chip and reader combination comprising
a) a biochip comprising one or several pluralities of ligand molecules for biomolecules attached to an essentially flat surface by means of covalent, quasicovalent, ionic, adhesive or adsorptive interaction, said essentially flat surface having the format of a 35 mm diapositive slide frame of 50 mm by 50 mm and
b) an electronic image capture system.

4. A combination according to claim 3 where the image capture system is a diapositive film slide scanner with electronic data capture.

5. A biochip comprising one or several pluralities of ligand molecules for biomolecules attached to an essentially flat surface by means of covalent, quasicovalent, ionic, adhesive or adsorptive interaction, said essentially flat surface having the format of a 35 mm diapositive slide frame of 50 mm by 50 mm.


SEQUENCE LISTING (5 sequences)

SEQ ID NO: 1 - DNA, 24 bp, Bos taurus
taacaacaatctccatgagttggt

SEQ ID NO: 2 - DNA, 24 bp, Sus scrofa
gataaaacataacttaacatggac

SEQ ID NO: 3 - DNA, 24 bp, Gallus gallus
gaccaccttacaaccttacacagc

SEQ ID NO: 4 - DNA, 25 bp, Sus scrofa
tgatccaacatcgaggtcgtaaacc

SEQ ID NO: 5 - DNA, 24 bp, Sus scrofa
aagacgagaagaccctrtggarct